(19) United States
(12) Patent Application Publication
(10) Pub. No.: US 2024/0417497 A1
(43) Pub. Date: Dec. 19, 2024

(54) SINTERED POROUS POLYPROPYLENE MEDIA AND APPLICATIONS

(71) Applicant: Porex Corporation, Fairburn, GA (US)

(72) Inventors: Shawn Patrick Knotts, Cumming, GA (US); Daniel George Fullerton, Cumming, GA (US); Michael Andrew Clendenning, Sharpsburg, GA (US); Serhan Yener, Suwanee, GA (US); Guoqiang Mao, Gilbert, AZ (US)

(21) Appl. No.: 18/737,697

(22) Filed: Jun. 7, 2024

Related U.S. Application Data
(60) Provisional application No. 63/472,658, filed on Jun. 13, 2023.

(51) Int. Cl. C08F 110/06; A61L 9/12; B01D 15/22; B01D 39/16; B01L 3/02; B43K 8/02; C08J 11/12

(52) U.S. Cl. CPC C08F 110/06; A61L 9/127; B01D 15/22; B01D 39/1661; B01L 3/0275; B43K 8/02; C08J 11/12; B01D 2239/1208; B01D 2239/1216; B01L 2300/0681; C08J 2323/12

(57) ABSTRACT

Compositions including a sintered porous ultra-high-molecular-weight polypropylene material having a viscosity average molecular weight over 500,000, an average porc size ranging from 10 μm to 200 μm, and a porosity ranging from 20% to 60%. Devices and methods including such compositions.

This application claims the benefit of U.S. Provisional Application No. 63/472,658, filed Jun. 13, 2023, and having a title of “Sintered Porous Polypropylene Media and Applications.” The contents of the above-identified Application is relied upon and incorporated herein by reference in its entirety.

### FIELD

Embodiments of the present invention are in the field of polymer processing and applications. In particular, embodiments relate to incorporating sintered porous thermoplastic media to various applications.

### BACKGROUND

Sintered porous polymeric materials find application and play critical roles in numerous fields. Sintered porous polymeric materials have been widely used in filtration, absorption, adsorption, venting, and fluid barrier applications. Currently, only limited sintered porous polymeric materials are available with wide range of pore size and porosity that meet the filtration, absorption, adsorption, venting, applying, wicking and liquid barrier requirements. As a result, components made of sintered porous polymeric materials are usually assembled with or into product constituents made of different polymeric material(s) to meet general application requirements of the product. However, products including assembled components and constituents of different polymeric materials make recycling difficult and expensive. Thus, it would be desirable for sintered porous polymeric constituents and non-porous constituents to use the same polymeric materials in an assembled component, product, or device.

### BRIEF SUMMARY

In view of the foregoing problems, it would be desirable to provide sintered porous polymeric compositions that share common polymeric compositions (e.g., same repeating subunit molecule) with non-porous constituents to improve the recyclability and sustainability of filtration, absorption, adsorption, applying, wicking, venting and fluid barrier products. In particular, polypropylene is one of the most widely available thermoplastics as filtration, absorption, adsorption, applying, wicking, venting, and fluid barrier media in filtration, absorption, adsorption, applying, wicking venting, and fluid barrier products and have desirable material properties over other widely available thermoplastics (e.g., polyethylene). When compared with other widely available thermoplastics, polypropylene is featured with high impact resistance, strong corrosion resistance, strong chemical stability, and strong thermal stability (e.g., higher melting point than polyethylene) that allows polypropylene to sustain a greater breadth of conditions (e.g., sterilization, autoclaving, etc.). Additionally, ultra-high-molecular-weight polypropylene (“UHMWPP”) with extremely long polymer chains and strengthened intermolecular interactions has improved material properties than regular polypropylene. By subjecting UHMWPP particles to a sintering process, the resulted sintered porous UHMWPP disclosed herein can achieve satisfactory porosity and average pore size as filtration, absorption, adsorption, applying, wicking, venting and fluid barrier media, thereby enabling more effective recycling of filtration, absorption, adsorption, applying, wicking, venting and fluid barrier polypropylene based products.

The present invention provides compositions including sintered porous polymeric materials so that the compositions have porosity and average pore size suitable as filtration, absorption, adsorption, applying, wicking, venting and fluid barrier media in various products. The present invention also provides apparatus comprising such compositions.

In an aspect, compositions are provided. An example composition of this aspect includes a sintered porous ultra-high-molecular-weight polypropylene material having a viscosity average molecular weight over 500,000, an average pore size ranging from 10 μm to 200 μm, and a porosity ranging from 20% to 60%.

In another aspect, pipette tips are provided. An example pipette tip includes a tubular tip defining a reservoir to receive a sample, wherein the tubular tip comprises polypropylene, and a porous thermoplastic plug mounted in the tubular tip, wherein the porous thermoplastic plug comprises a sintered porous ultra-high-molecular-weight polypropylene material having a viscosity average molecular weight over 500,000, an average pore size ranging from 10 μm to 200 μm, and a porosity ranging from 20% to 60%.

In another aspect, solid phase extraction columns are provided. An example solid phase extraction column includes a barrel defining a sample reservoir, wherein the barrel comprises polypropylene, and a plurality of frits disposed in the barrel, wherein the plurality of frits comprises a sintered porous ultra-high-molecular-weight polypropylene material having a viscosity average molecular weight over 500,000, an average pore size ranging from 10 μm to 200 μm, and a porosity ranging from 20% to 60%.

In another aspect, multi-well devices are provided. An example multi-well device includes a housing including a plurality of wells, wherein the housing and the plurality of wells comprise polypropylene, and a plurality of filters disposed in the housing, wherein the plurality of filters comprises a sintered porous ultra-high-molecular-weight polypropylene material having a viscosity average molecular weight over 500,000, an average pore size ranging from 10 μm to 200 μm, and a porosity ranging from 20% to 60%.

In another aspect, writing instruments are provided. An example writing instrument includes a barrel containing a solution, wherein the barrel comprises polypropylene, and a nib connected to the barrel, wherein the nib comprises a sintered porous ultra-high-molecular-weight polypropylene material having a viscosity average molecular weight over 500,000, an average pore size ranging from 10 μm to 200 μm, and a porosity ranging from 20% to 60%.

In another aspect, liquid applicators are provided. An example liquid applicator includes a housing containing a solution, wherein the housing comprises polypropylene, and a filter disposed in the housing, wherein the filter comprises a sintered porous ultra-high-molecular-weight polypropylene material having a viscosity average molecular weight over 500,000, an average pore size ranging from 10 μm to 200 μm, and a porosity ranging from 20% to 60%.

In another aspect, liquid emanation devices are provided. An example liquid emanation device includes a housing containing a solution, wherein the housing comprises polypropylene, and a wick in fluidic connection with the solution and disposed in the housing, wherein the wick comprises a sintered porous ultra-high-molecular-weight polypropylene material having a viscosity average molecular weight over 500,000, an average pore size ranging from 10 μm to 200 μm, and a porosity ranging from 20% to 60%. In some examples, the solution comprises a fragrance or an insecticide.

In another aspect, devices including compositions described above are provided. The example devices include a liquid collection device including a polypropylene housing and a filter including the composition described herein, and a diagnostic device comprising a polypropylene housing and a component including the composition described herein.

In another aspect, an example method of recycling is provided. The example method includes reprocessing a device by melting a device without separating device components, wherein the device components include a housing comprising polypropylene and a functional component comprising a sintered porous ultra-high-molecular-weight polypropylene material having a viscosity average molecular weight over 500,000, an average pore size ranging from 10 μm to 200 μm, and a porosity ranging from 20% to 60%.

In another aspect, methods of recycling are provided. An example method of recycling includes disposing a recyclable article in a recycle chamber, wherein the recyclable article is a thermoplastic, wherein the recyclable article includes a component comprising sintered polymeric material that is porous, wherein each component of the recyclable article comprises a same repeating subunit molecule, depolymerizing the recyclable article by heating the recycle chamber to obtain a plurality of the subunit molecules, purifying the plurality of the subunit molecules to obtain a plurality of purified subunit molecules, and repolymerizing the plurality of purified subunit molecules to form a recycled polymer. In some examples, the sintered polymeric material has a porosity ranging from 20% to 60%, wherein the sintered polymeric material is polypropylene, and the sintered polymeric material has a viscosity average molecular weight over 500,000 or 1,000,000.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 shows a sintering process of ultra-high-molecular-weight polypropylene according to some examples.

FIG. 2 shows schematic illustrations of a pipette tip including a porous plastic plug having a sintered porous ultra-high-molecular-weight polypropylene material according to some examples.

FIG. 3 shows schematic illustrations of a writing instrument including a sintered porous polymeric material nib having a sintered porous ultra-high-molecular-weight polypropylene material according to some examples.

FIG. 4 shows simplified schematic illustrations of a tip portion of a writing instrument including a sintered porous ultra-high-molecular-weight polypropylene nib according to some examples.

FIG. 5 shows schematic illustrations of a liquid emanation device including a sintered porous ultra-high-molecular-weight polypropylene wick according to some examples.

### DETAILED DESCRIPTION

Currently available sintered porous polymeric materials as filtration, absorption, adsorption, applying, wicking, venting, and fluid barrier media are limited to polyethylene, especially ultra-high-molecular weight polyethylene (“UHMWPE”). However, most polymeric devices including filtration and fluid barrier media uses housings made from polypropylene. Since polypropylene and polyethylene (especially UHMWPE) are non-compatible materials, they could not be reprocessed together as a whole piece. Additionally, the cost of separating components made of different materials (e.g., polypropylene, polyethylene, UHMWPE, etc.) is usually too high to be practicable. As mentioned earlier, many polymeric devices (e.g., pipette tips, solid phase extraction columns, multi-well devices, writing instruments, liquid applicators, etc.) include housing made from polypropylene and filtration and fluid barrier media made from polyethylene. To avoid the high cost of separating components such as housing and barrier media made of different materials, those polymeric devices (e.g., pipette tips, solid phase extraction columns, multi-well devices, writing instruments, liquid emanation devices, liquid applicators, etc.) are usually disposed as landfills, which causes environment issues and concerns. If the sintered porous filtration, absorption, adsorption, applying, wicking, venting, and fluid barrier media in these polypropylene based devices are made from ultra-high-molecular weight polypropylene, the whole device could be remelted and reprocessed into new products without separating the device components. Consequently, the devices will not go into landfills and cause environment issues.

A better understanding of the nature and advantages of embodiments of the present invention may be gained with reference to the following detailed description and the accompanying drawings.

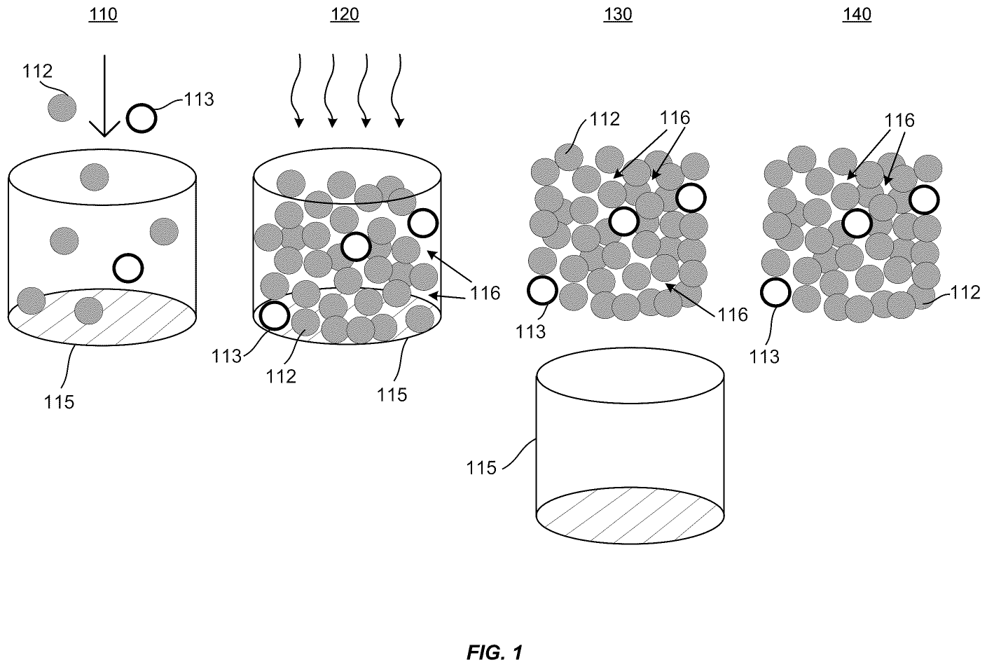

FIG. 1 shows a sintering process of ultra-high-molecular-weight polypropylene according to some examples. Similar to sintering processes for polyethylene and/or UHMWPE, the example sintering process includes compacting and forming a solid mass of porous material by heating a mixture of sintering particles without fully melting the mixture of sintering particles. In other words, the sintering particles are heated to be softened without being melted. For the invention disclosed herein, the mixture of sintering particles includes a plurality of ultra-high-molecular-weight polypropylene (“UHMWPP”) particles 112. At the initial stage 110, a mixture of sintering particles (e.g., a plurality of UHMWPP particles 112) is placed in a mold 115 that maintains the mixture of sintering particles in a desired shape or geometry of a final product. The plurality of UHMWPP particles 112 may be in any form as desired. In some examples, the plurality of UHMWPP particles 112 may be in the form of powder. In some examples, the plurality of UHMWPP particles 112 in the form of powder may be characterized by an average of size of particles. In some examples, the plurality of UHMWPP particles 112 in the form of powder may have an average particle size ranging from 10 μm to 300 μm. For examples, the average particle size may be in ranges of 10-50 μm, 50-100 μm, 100-150 μm, 150-200 μm, 200-250 μm, and/or 250-300 μm. In some examples, the UHMWPP may have a viscosity average molecular weight over 500,000. For examples, the viscosity average molecular weight may be in ranges of 500,000-600,000, 600,000-700,000, 700,000-800,000, 800,000-900,000, 900,000-1,000,000, 1,000,000-1,100,000, 1,100,000-1,200,000 or above.

In some examples, the mixture of sintering particles may optionally include a plurality of additive particles 113 that are intentionally added to the mixture of the sintering particles to control the microstructural and dimensional development during sintering. In some examples, the plurality of additive particles 113 may also modify hydrophilicity, hydrophobicity, absorption, adsorption, recyclability, and/or color-changeability of the mixture of sintering particles or any resulting substance from the sintering process. In some examples, the additives may be filters or catalysts that absorbs undesired gas. In some examples, the additives may facilitate the preservation of porosity during the sintering process. In some examples, the additives may include an absorbent that inhibits or prevents liquid from traveling through. In some examples, the absorbent may include carboxymethylcellulose (“CMC”), cellulose gums, hydrolyzed acrylonitrile graft copolymer, neutralized starch-acrylic acid graft copolymer, acrylamide copolymer, modified crosslinked polyvinyl alcohol, neutralized self-crosslinking polyacrylic acid, crosslinked polyacrylate salts, or neutralized crosslinked isobutylene-maleic anhydride copolymers, or salts or mixtures thereof. In some examples, the additives may include color change indicator comprises a dye, including, but not limited to, inorganic or organic dyes, such as food dyes, azo compounds, or azo dyes. In some examples, the additives may include pigments. Details related to porous barrier media comprising color change indicators are illustrated by commonly owned U.S. Pat. No. 8,187,534 entitled “POROUS BARRIER MEDIA COMPRISING COLOR CHANGE INDICATORS” to Mao et al, owned by the common assignee of the present disclosure, and hereby incorporated by reference in its entirety for all purposes. The additives may be in any form as desired. In some examples, the additives may be in the form of powder. In some examples, the plurality of additive particles 113 may include activated carbon particles.

At the first intermediate stage 120, the mixture of sintering particles including the plurality of UHMWPP particles 112 is heated to a sintering temperature at which the plurality of UHMWPP particles 112 becomes softened but not melted. As discussed above, the mixture of sintering particles may optionally include a plurality of additive particles 113. As the softened UHMWPP particles 112 become fused into a dense bulk, a plurality of pores 116 are formed. The heating of the mixture of sintering particles continues until the sintered bulk with satisfactory properties (e.g., porosity, pore size) is obtained. In other examples, the properties may be any material property that includes, but is not limited to, composition, density, melting point, strength, electrical conductivity, translucency, thermal conductivity, the uniformity of pore size, etc.

At the second intermediate stage 130, the sintered bulk is separated from the mold 115. As illustrated by FIG. 1, the sintered bulk may include the plurality of UHMWPP particles 112 and the plurality of pores 116 as desired. Optionally, the sintered bulk may also include the plurality of additive particles 113.

At the final stage 140, the sintered bulk may be optionally prepared with any means or methods as needed so that the sintered bulk is in a condition suitable for any downstream processing. The sintered bulk at the final stage 140 may be referred to as a “final sintered bulk.” In some examples, the final sintered bulk may have a porosity ranging from 20% to 60%. For example, the porosity may be from 20% to 25%, 25% to 30%, 30% to 35%, 35% to 40%, 40% to 45%, 45% to 50%, 50% to 55%, or 55% to 60%. In some examples, the final sintered bulk may have an average pore size ranging from 10 μm to 200 μm. In some examples, the final sintered bulk may have a bulk density that ranges in 0.2-0.3 g/ml, 0.3-0.4 g/ml, and/or 0.4-0.5 g/ml. In some examples, the final sintered bulk may have a melting temperature of at least 150° C. In other examples, the final sintered bulk may have a higher softening or melting temperature to sustain a desired sterilization condition than polyethylene products. In some examples, the sintered bulk has an infinitesimal melting flow index at a temperature of 230° C. under a 21.6 kg load with a loading time of 10 minutes. In some examples, the sintered bulk has a flow index that ranges from 0-1, 1-2, 2-3, 3-4, 4-5, and 5-6 kg/230° C. under a 21.6 kg load with a loading time of 10 minutes. In some examples, the sintered bulk may exclude polyethylene. In some examples, the sintered bulk may include a flexible region and a rigid region.

The final sintered bulk or the composition as described in relation to FIG. 1 may be incorporated into various apparatuses as desired. The final sintered bulk or the composition may be a sintered porous UHMWPP material having a viscosity average molecular weight over 500,000, an average pore size ranging from 10 μm to 200 μm, and a porosity ranging from 20% to 60%. Example apparatuses include, but are not limited to, a multi-well device including a polypropylene housing that further includes a plurality of polypropylene wells and a plurality of filters disposed in the housing and made of the sintered porous UHMWPP material, a liquid applicator including a housing made of polypropylene and a nib made of the sintered porous UHMWPP material, a liquid collection device including a polypropylene housing and a filter made of the sintered porous UHMWPP material, or a diagnostic device including a polypropylene housing and a component including the sintered porous UHMWPP material.

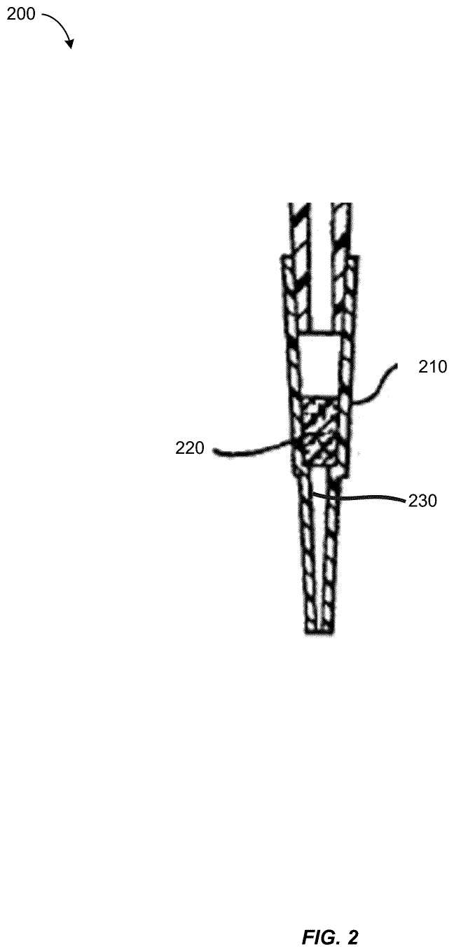

FIG. 2 shows schematic illustrations of a pipette tip including a porous plastic plug having a sintered porous ultra-high-molecular-weight polypropylene material according to some examples. The pipette device 200 includes a pipette tip 210 in which is mounted a porous plastic plug 220. In some examples, the pipette tip 210 may include a tubular tip 230 defining a reservoir to receive a sample. In some examples, the pipette tip 210 and the tubular tip 230 may include polypropylene. In some examples, the porous plastic plug 220 may include a sintered porous ultra-high-molecular-weight polypropylene material that has a viscosity average molecular weight over 500,000, an average pore size ranging from 10 μm to 200 μm, and a porosity ranging from 20% to 60%. The pipette tip 210 may serve as a liquid and/or aerosol barrier.

In addition to the description set forth herein, further details of a pipette device can be found in commonly owned U.S. Pat. No. 5,364,595 entitled “PIPETTE DEVICE CONSTRUCTED TO PREVENT CONTAMINATION BY AEROSOLS OR OVERPIPETTING” to Smith, owned by the common assignee of the present disclosure, and hereby incorporated by reference in its entirety for all purposes.

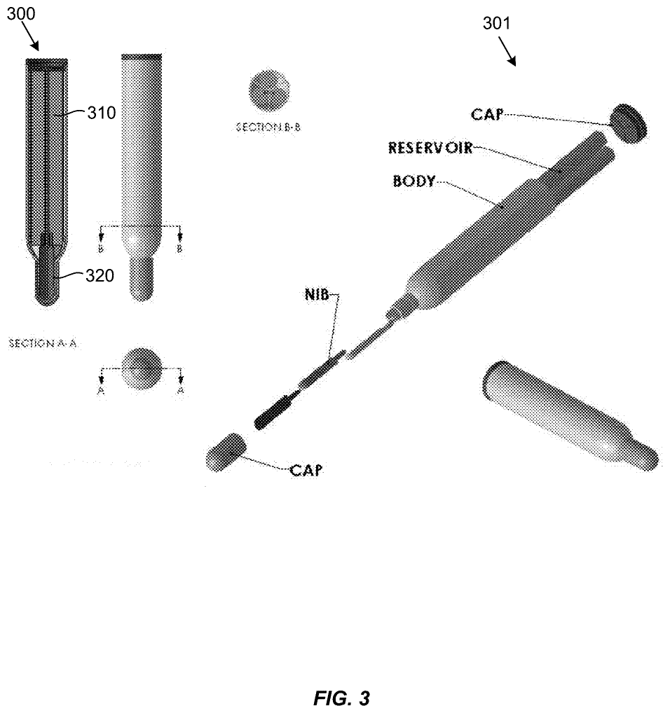

FIG. 3 shows schematic illustrations of a writing instrument including a sintered porous polymeric material nib having a sintered porous ultra-high-molecular-weight polypropylene material according to some examples. The use of the term “pen” in this invention is not intended to refer to a device for applying ink on a surface as a writing instrument but is instead used to refer to a device which may be held like a pen and is used as a sample collection device. The term “pen” may be used interchangeably with the term “writing instrument.” As illustrated by FIG. 3, a writing instrument 300 may include a barrel 310 containing a solution and a nib 320 that is connected to the barrel 310. In some examples, the barrel 310 may include or be made of a first polymeric material. The nib 320 may be made of a second polymeric material and the second polymeric material may be a sintered porous polymeric material. In some examples, the sintered porous polymeric material included in the nib 320 is formed by heating polypropylene particles having a particle size less than 300 μm. In some examples, the first polymeric material and the second polymeric material include the same repeating subunit molecule (e.g., propylene). The nib 320 (for collecting the sample in some examples) may be attached or otherwise secured at one or both ends and is contained in the barrel 310. The nib 320 may be friction fit into the barrel 310, such that the nib 320 may be pressed into an opening at an end of the writing instrument 300 and remain securely connected thereto. In some examples, the nib 320 may be secured to (or formed with) a nib stem, which may then be friction fit into the barrel 310. For example, the nib and/or the nib stem may have a geometry and shape that matches an internal geometry of the barrel 310 and shape of the writing instrument 300, such that the nib 320 and/or nib stem can be securely connected within the barrel 310. The nib stems may have cross-sectional shapes that are generally round, square, flat or rectangular, star shaped, or any other appropriate shape. It is possible in this and other embodiments to provide a protrusion at the end of the nib stem that fits into a recess of the barrel 310 or vice versa, such that an additional male/female connection secures the nib 320 and/or nib stem in place. Examples of alternate nib stem shapes and writing instruments are illustrated by commonly owned U.S. Pat. No. 8,852,122 entitled “LIQUID SAMPLING, STORAGE, TRANSFER AND DELIVERY DEVICE” to Mao et al, owned by the common assignee of the present disclosure, and hereby incorporated by reference in its entirety for all purposes. Examples and details of alternate nibs and sintered polymeric materials are illustrated by commonly owned U.S. Pat. No. 8,141,717 entitled “SINTERED POLYMERIC MATERIALS AND APPLICATIONS THEREOF” to Wingo et al, owned by the common assignee of the present disclosure, and hereby incorporated by reference in its entirety for all purposes.

As shown by illustration 301, in some examples, the barrel 310 may have a body, a reservoir, and a cap. The reservoir may be provided as a separate element or the reservoir may be built into a pen body. In some examples, all constituents, components, and parts of the writing instrument 300 include polypropylene or ultra-high-molecular-weight polyethylene (“UHMWPP”) so that the writing instrument 300 can be recycled without incurring additional preparation, sorting, or disassembling steps.

In some examples, the nib 320 may include sintered porous UHMWPP having a viscosity average molecular weight over 500,000. For examples, the viscosity average molecular weight may be in ranges of 500,000-600,000, 600,000-700,000, 700,000-800,000, 800,000-900,000, 900,000-1,000,000, 1,000,000-1,100,000, 1,100,000-1,200,000 or above. In some examples, the UHMWPP powder particles being used to fabricate the sintered porous UHMWPP may have an average size ranging from 10 to 300 μm. In some examples, the sintered porous UHMWPP may have an average pore size that ranges from 10 μm to 200 μm. In some examples, the sintered porous UHMWPP may have a porosity ranging from 20% to 60%. In some examples, the the sintered porous UHMWPP may have an infinitesimal melting flow index at a temperature of 230° C. under a 21.6 kg load with a loading time of 10 minutes. In some examples, the sintered porous UHMWPP may have a flow index that ranges from 0-1, 1-2, 2-3, 3-4, 4-5, and 5-6 kg/230° C. under a 21.6 kg load with a loading time of 10 minutes. In some examples, the sintered porous UHMWPP may further include activated carbon particles. In some examples, the sintered porous UHMWPP may further include surfactant.

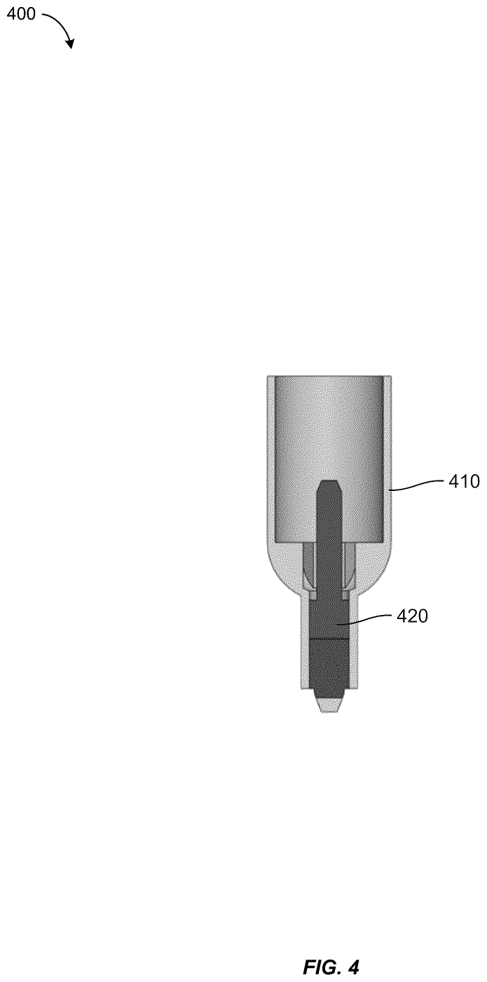

FIG. 4 shows simplified schematic illustrations of a tip portion of a writing instrument including a sintered porous ultra-high-molecular-weight polypropylene nib according to some examples. The tip portion 400 of the writing instrument may include a nib 420 that is connected to a barrel 410. The barrel 410 is made of polypropylene. The nib 420 is made of a sintered porous ultra-high-molecular-weight polypropylene (“UHMWPP”) material. The sintered porous UHMWPP material may have a viscosity average molecular weight over 500,000. The sintered porous UHMWPP material has an average pore size ranging from 10 μm to 200 μm and a porosity ranging from 20% to 60%. In some examples, the sintered porous UHMWPP material is fabricated from UHMWPP powder having an average particle size ranging from 10 μm to 300 μm.

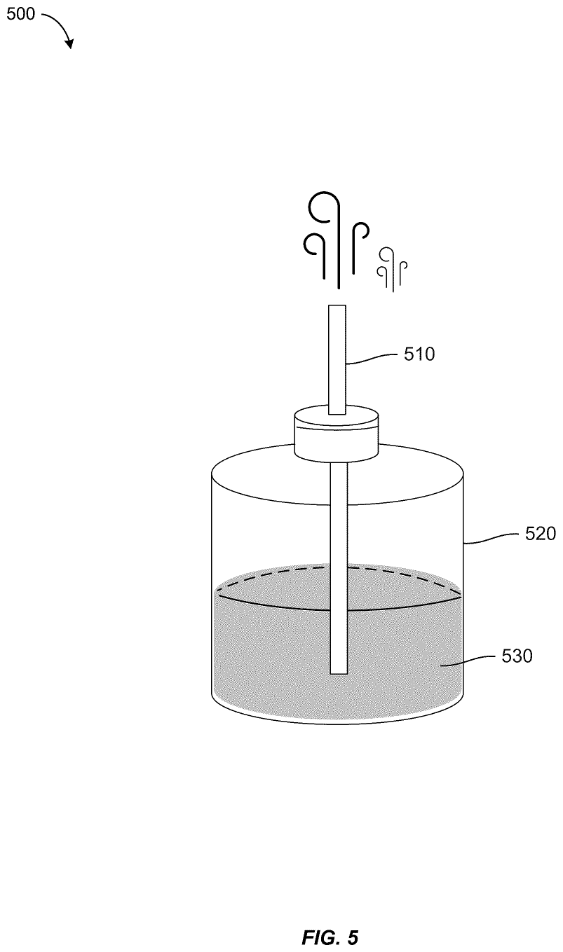

FIG. 5 shows schematic illustrations of a liquid emanation device including a sintered porous ultra-high-molecular-weight polypropylene wick according to some examples. In some examples, the liquid emanation device may be a fragrance emanation device or an insecticidal emanation device. The liquid emanation device 500 includes a wick 510 in fluidic connection to a solution 530 and a housing 520 in which the wick 510 is disposed. The wick 510 is made of a sintered porous ultra-high-molecular-weight polypropylene (“UHMWPP”) material. The sintered porous UHMWPP material has an average pore size ranging from 10 μm to 200 μm and a porosity ranging from 20% to 60%. The sintered porous UHMWPP material may have a viscosity average molecular weight over 500,000. In some examples, the sintered porous UHMWPP material is fabricated from UHMWPP powder having an average particle size ranging from 10 μm to 300 μm. The housing 520 is made of a polypropylene material. In some examples, the solution 530 may include fragrances or insecticides.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

What is claimed is:

1. A composition comprising:
a sintered porous ultra-high-molecular-weight polypropylene material having a viscosity average molecular weight over 500,000, an average pore size ranging from 10 μm to 200 μm, and a porosity ranging from 20% to 60%.

2. A pipette tip, comprising:
a tubular tip defining a reservoir to receive a sample, wherein the tubular tip comprises polypropylene; and
a porous thermoplastic plug mounted in the tubular tip, wherein the porous thermoplastic plug comprises a sintered porous ultra-high-molecular-weight polypropylene material having a viscosity average molecular weight over 500,000, an average pore size ranging from 10 μm to 200 μm, and a porosity ranging from 20% to 60%.

3. A solid phase extraction column, comprising:
a barrel defining a sample reservoir, wherein the barrel comprises polypropylene; and
a plurality of frits disposed in the barrel, wherein the plurality of frits comprises a sintered porous ultra-high-molecular-weight polypropylene material having a viscosity average molecular weight over 500,000, an average pore size ranging from 10 μm to 200 μm, and a porosity ranging from 20% to 60%.

4. A multi-well device, comprising:
a housing including a plurality of wells, wherein the housing and the plurality of wells comprise polypropylene; and
a plurality of filters disposed in the housing, wherein the plurality of filters comprises a sintered porous ultra-high-molecular-weight polypropylene material having a viscosity average molecular weight over 500,000, an average pore size ranging from 10 μm to 200 μm, and a porosity ranging from 20 to 60%.

5. A writing instrument, comprising:
a barrel containing a solution, wherein the barrel comprises polypropylene; and
a nib connected to the barrel, wherein the nib comprises a sintered porous ultra-high-molecular-weight polypropylene material having a viscosity average molecular weight over 500,000, an average pore size ranging from 10 μm to 200 μm, and a porosity ranging from 20% to 60%.

6. A liquid applicator, comprising:
a housing containing a solution, wherein the housing comprises polypropylene; and
a filter disposed in the housing, wherein the filter comprises a sintered porous ultra-high-molecular-weight polypropylene material having a viscosity average molecular weight over 500,000, an average pore size ranging from 10 μm to 200 μm, and a porosity ranging from 20% to 60%.

7. A liquid emanation device, comprising:
a housing containing a solution, wherein the housing comprises polypropylene; and
a wick in fluidic connection with the solution and disposed in the housing, wherein the wick comprises a sintered porous ultra-high-molecular-weight polypropylene material having a viscosity average molecular weight over 500,000, an average pore size ranging from 10 μm to 200 μm, and a porosity ranging from 20% to 60%.

8. The emanation device of claim 7, wherein the solution comprises a fragrance or an insecticide.

9. A device comprising the composition of claim 1.

10. The device of claim 9, wherein the device is a liquid collection device comprising a polypropylene housing and a filter comprising the composition of claim 1 or a diagnostic device comprising a polypropylene housing and a component comprising the composition of claim 1.

11. A method of recycling, comprising:
reprocessing a device by melting a device without separating device components, wherein the device components include a housing comprising polypropylene and a functional component comprising a sintered porous ultra-high-molecular-weight polypropylene material having a viscosity average molecular weight over 500,000, an average pore size ranging from 10 μm to 200 μm, and a porosity ranging from 20% to 60%.

12. A method of recycling, comprising:
disposing a recyclable article in a recycle chamber, wherein the recyclable article is a thermoplastic, wherein the recyclable article includes a component comprising sintered polymeric material that is porous, wherein each component of the recyclable article comprises a same repeating subunit molecule;
depolymerizing the recyclable article by heating the recycle chamber to obtain a plurality of subunit molecules;
purifying the plurality of the subunit molecules to obtain a plurality of purified subunit molecules; and
repolymerizing the plurality of purified subunit molecules to form a recycled polymer.

13. The method of claim 12, wherein the sintered polymeric material has a porosity ranging from 20% to 60%, wherein the sintered polymeric material is polypropylene, and the sintered polymeric material has a viscosity average molecular weight over 500,000 or 1,000,000.

14. The method of claim 12, wherein the recyclable article is any of the pipette tip of claim 2, the multi-well device of claim 4, the writing instrument of claim 5, the liquid applicator of claim 6, the liquid collection device or the diagnostic device of claim 10.
